(19) United States
(12) Patent Application Publication
(10) Pub. No.: US 2023/0258655 A1
(43) Pub. Date: Aug. 17, 2023

(54) KIT FOR DETECTING DUST MITE COMPONENT-SPECIFIC ANTIBODIES

(71) Applicant: HANGZHOU ZHEDA DIXUN BIOLOGICAL GENE ENGINEERING CO., LTD., Hangzhou, Zhejiang (CN)

(72) Inventors: Shandong WU, Hangzhou (CN); Huahao SHEN, Hangzhou (CN); Yi LIU, Hangzhou (CN); Zhoujie WU, Hangzhou (CN); Suling XU, Hangzhou (CN); Wei LEI, Hangzhou (CN); Yifei WANG, Hangzhou (CN); Puyang XU, Hangzhou (CN); Xiandong ZHOU, Hangzhou (CN); Weiyue CAI, Hangzhou (CN); Mingzhi ZHU, Hangzhou (CN); Xukai YANG, Hangzhou (CN)

(73) Assignee: HANGZHOU ZHEDA DIXUN BIOLOGICAL GENE ENGINEERING CO., LTD., Hangzhou, Zhejiang (CN)

(21) Appl. No.: 18/027,624

(22) PCT Filed: Jun. 10, 2021
(86) PCT No.: PCT/CN2021/099383

§ 371 Date: Mar. 21, 2023

(30) Foreign Application Priority Data
Apr. 16, 2021 (CN) 202110409090.X

(51) Int. Cl. G01N 33/68; G01N 33/543; G01N 33/547

(52) U.S. Cl. CPC G01N 33/6854; G01N 33/54306; G01N 33/547; G01N 2333/43582; G01N 2800/24

(57) ABSTRACT

The present disclosure relates to a kit for detecting dust mite component-specific antibodies, and belongs to the technical field of antibody detection kits. The kit comprises a biotin-polystreptavidin-biotin-dust mite antigen-coated nitrocellulose (NC) membrane, a washing solution, an alkaline phosphatase (ALP)-labeled secondary antibody solution for dust mite component-specific antibodies, and a substrate solution. The kit may be used in cooperation with a fully automated instrument.

### CROSS-REFERENCE TO RELATED APPLICATION

This patent application claims the benefit and priority of Chinese Patent Application No. 202110409090.X, entitled “KIT FOR DETECTING DUST MITE COMPONENT-SPECIFIC ANTIBODIES” filed with the China National Intellectual Property Administration on Apr. 16, 2021, the disclosure of which is incorporated by reference herein in its entirety as part of the present application.

### TECHNICAL FIELD

The present disclosure relates to the technical field of allergen detection kits, and in particular to a kit for detecting dust mite component-specific antibodies.

### BACKGROUND ART

Mites are ubiquitous creatures, which cannot be seen without a microscope due to their small size. In fact, mites can be found in various natural conditions or human living environments. At present, in allergology, the most noteworthy mites are mites in the human living environment that can induce human allergic reactions. With the modernization of human living environment, an incidence of allergic diseases related to mites has increased rapidly. These mites mainly include dust mites and storage mites. The dust mites mainly include Dermatophagoides farinae, Dermatophagoides pteronyssinus, Euroglyphus maynei, and Acarus siro. The storage mites mainly include Blomia tropicalis. The most common mites in living rooms worldwide are Dermatophagoides farinae and Dermatophagoides pteronyssinus. Dust mite allergy is one of the most common allergic diseases, which can be manifested as allergic rhinitis, allergic asthma, atopic dermatitis, allergic conjunctivitis, gastrointestinal dysfunction, and the like.

IgE-mediated type I hypersensitivity diseases (i.e., allergic diseases) are quite common. The pathogenesis of allergic diseases is as follows: when a patient inhales or ingests an allergenic ingredient-containing substance (called allergen), B cells in the body are triggered to produce immunoglobulin E (IgE), and the Fc fragment of IgE binds to the corresponding FccRI on the surface of mast cells (MCs) or basophils to make the body in a state of sensitization to the allergen; the allergen, when entering the sensitized body once again, can specifically bind to IgE on the surface of MCs or basophils; when the allergen binds to two or more adjacent IgE on the surface of the sensitized cells, FccRI cross-linking occurs, which activates MCs and basophils; and the activation causes the cells to degranulate and release histamine stored in cytoplasmic granules and to synthesize leukotrienes, immunoreactive prostaglandins, and IL4, IL5 and other cytokines and chemokines through the arachidonic acid pathway, which trigger anaphylaxis (or allergy). IgE plays a key role in the occurrence of allergic diseases. Allergic diseases are characterized by a higher allergen-specific IgE (sIgE) concentration in the circulating blood of a patient than that under normal conditions, and the higher the sIgE concentration, the higher the probability of suffering from allergic diseases.

In recent years, allergen-specific immunotherapy (SIT) has been widely used in the treatment of allergic rhinitis, asthma, and other allergic diseases caused by dust mites. SIT is the only etiological treatment method that can affect the natural course of allergic diseases (Jean, Bousquet, and, et al. Allergen immunotherapy: Therapeutic vaccines for allergic diseases A WHO position paper [J]. Journal of Allergy & Clinical Immunology, 1998). SIT refers to a treatment method described as follows: after an allergen causing an allergic reaction in a patient is determined, a high-purity standardized allergen vaccine is used to strengthen the patient's tolerance to the antigen (with a concentration and a dosage increasing progressively from a low concentration and a small dosage), such that the patient will not have the anaphylaxis or show alleviated symptoms when exposed to the allergen once again.

IgG4 is a subtype of IgG (Immunoglobulin G, IgG), and sIgG/sIgG4 acts as a “blocking antibody” in the therapeutic process of SIT. sIgG/sIgG4 can hinder the synthesis of IgE, reduce the amount of IgE, compete with IgE for Fc receptors on the surface of MCs, basophils, etc., prevent the occurrence of IgE-mediated MC degranulation, and reduce the release of histamine, leukotrienes, and other inflammatory mediators, such that the airway inflammation in children with asthma, lung function, and asthma symptoms can be improved and the airway hyperresponsiveness can be reduced. After a period of SIT treatment, a long-term therapeutic effect is available for the body even if the treatment is stopped. In vitro experiments also show that the antibody can reduce allergen-specific T cell responses by inhibiting the binding of allergen-IgE complexes to antigen-presenting cells (APCs). A successful SIT is usually accompanied by a significant increase of serum sIgG/sIgG4. Studies have shown that different populations show different responses to dust mite allergen components, and different allergens lead to different clinical effects when used for SIT (M Guimares Junqueir de Queirós, Silva D A O, Alves R, et al. Mite-specific immunotherapy using allergen and/or bacterial extracts in atopic patients in Brazil[J]. J Investig Allergol Clin Immunol, 2007, 18(2): 84-92.).

In allergen component diagnosis, monomeric allergens are used to identify specific molecules causing allergies, which can improve the accuracy of allergy diagnosis and desensitization treatment. In terms of identification and molecular diagnosis of allergens, the current research and industrialization levels in China show a gap from that of developed countries in Europe and America, which is manifested in depth and breadth. So far, there are very few allergen components that can be used industrially and there is no kit for allergen diagnosis in China. At present, there are few allergen component diagnosis products with incomplete items worldwide, which have not been approved for marketing in China. For example, the ImmunoCAP for dust mite component detection from Thermo Fisher can only detect 4 dust mite components: Der p 1, Der p 2, Der p 10, and Der p 23, and cannot detect antibodies specific for other dust mite components. Moreover, the ImmunoCAP has a low throughput, and one CAP can only detect one component. These are not conducive to the development of individualized dust mite component SIT.

Therefore, the detection of various dust mite component-specific antibodies (sIgE) can help to determine the specific allergic component for an allergic patient, which is conducive to the more accurate determination of dust mite allergy; the detection of various dust mite component-specific antibodies sIgG/sIgG4 can provide a basis for accurate immunotherapy of patients and play an indicative role in subsequent dust mite desensitization treatment.

However, there is currently no kit for efficiently detecting allergen components and dust mite component-specific antibodies.

### SUMMARY

The present disclosure is intended to provide a kit for detecting dust mite component-specific antibodies. The kit of the present disclosure has reliable performance, high sensitivity, and wide linear range, and can be used in combination with full-automatic instruments, which, may improve detection sensitivity and reliability and reduce cost. Moreover, the kit of the present disclosure may efficiently detect dust mite allergen components and various dust mite component-specific antibodies including IgE, sIgG and sIgG4, with high throughput.

The present disclosure provides a kit for detecting dust mite component-specific antibodies, including: a biotin-polystreptavidin-biotin-dust mite antigen-coated nitrocellulose (NC) membrane, a washing solution, an alkaline phosphatase (ALP)-labeled secondary antibody solution for dust mite component-specific antibodies, and a substrate solution,

where the biotin-polystreptavidin-biotin-dust mite antigen-coated NC membrane is formed by coating an NC membrane sequentially with solidified biotin, polystreptavidin, and biotin-labeled dust mite antigens; the dust mite antigens include: dust mite protein Der f 1, dust mite protein Der f 2, dust mite protein Der p 1, dust mite protein Der p 2, dust mite protein Der p 5, dust mite protein Der p 7, dust mite protein Der p 10, dust mite protein Der p 11, dust mite protein Der p 20, dust mite protein Der p 21, dust mite protein Der p 23, dust mite protein Blo t 5, dust mite protein Blo t 10, dust mite protein Blo t 21, Dermatophagoides pteronyssinus, Dermatophagoides farinae, and Blomia tropicalis; and the dust mite antigens are coated on different positions on the NC membrane.

The dust mite component-specific antibodies include sIgE and sIgG, and the sIgG includes sIgG4.

In some embodiments, a preparation method of the biotin-polystreptavidin-biotin-dust mite antigen-coated NC membrane may include the following steps: coating biotin on an NC membrane, and drying; coupling polystreptavidin to the biotin, and drying; and coupling biotin-labeled dust mite antigens to the polystreptavidin.

In some embodiments, the washing solution may be a Tris-HCl buffer containing Tween-20.

In some embodiments, the Tween-20 may have a mass percentage of 0.0004% to 0.02% in the washing solution, and the washing solution may have a pH of 7.2 to 7.6.

In some embodiments, a preparation method of the ALP-labeled secondary antibody solution for dust mite component-specific antibodies may include:

mixing ALP with a secondary antibody for dust mite component-specific antibodies to obtain the ALP-labeled secondary antibody solution for dust mite component-specific antibodies.

In some embodiments, the secondary antibody solution for dust mite component-specific antibodies may have a mass concentration of 0.1 μg/mL to 10 μg/mL.

In some embodiments, the substrate solution may be a BCIP/NBT substrate solution.

The present disclosure further provides a method for high-throughput detection of dust mite antigens and single-component sIgE or sIgG (including IgG4) using the kit of the present disclosure may preferably include the following steps:

wetting a detection plate with the working washing solution; adding test serum, and incubating; washing, adding the ALP-labeled secondary antibody solution for dust mite component-specific antibodies, and incubating; washing, adding the substrate solution, and incubating; and stopping the reaction, drying, and scanning with a scanner.

The present disclosure provides a kit for detecting dust mite component-specific antibodies. In the present disclosure, a variety of major standardized dust mite allergen components are used to develop a dust mite component-specific IgE and IgG (including IgG4) detection kit suitable for actual conditions in China. The kit can quantitatively detect dust mite component-specific antibodies (sIgE and sIgG (including IgG4)) levels in human serum, which clinically can be used for in vitro auxiliary diagnosis of type I hypersensitivity diseases and play an indicative role in dust mite desensitization treatment. In the present disclosure, the high-throughput protein microarray technology is used to coat an NC membrane with dust mite allergen proteins: Der f 1, Der f 2, Der p 1, Der p 2, Der p 5, Der p 7, Der p 10, Der p 11, Der p 20, Der p 21, Der p 23, Blot 5, Blot 10, and Blo t 21, and 3 dust mite allergens: Dermatophagoides pteronyssinus, Dermatophagoides farinae, and Blomia tropicalis, such that 14 dust mite allergen proteins, altogether 17 items, can be detected at one time.

The kit of the present disclosure adopts the amplification structure of biotin-polystreptavidin-biotin-antigen, such that an antigen can fully react with an antibody to improve the detection sensitivity. In the present disclosure, single purified natural or recombinant dust mite allergen components are used to identify specific molecules causing allergies, which may improve the accuracy of allergy diagnosis and prognosis; and a specific allergic component can be detected for a patient, which may be conducive to accurate determination of dust mite allergies and provides a basis for accurate immunotherapy on patients. the present disclosure can quantitatively detect the levels of dust mite component-specific IgG (sIgG), and specific IgG4 (sIgG4) antibodies in human serum during desensitization treatment, which may clinically be used for in vitro auxiliary diagnosis of the body's immune responses to dust mite antigen exposure; and the present disclosure may also detect the specific components causing immune responses in the patient, which may provide a basis for accurate immunotherapy of patients and play an indicative role in subsequent dust mite desensitization treatment. The kit of the present disclosure may be used subsequently in combination with scanning analysis software to accurately identify protein sites and effectively determine sIgE and sIgG (including IgG4) concentrations, which may improve the accuracy.

### BRIEF DESCRIPTION OF DRAWINGS

FIG. 1 shows detection results of the sIgG4 content provided by the present disclosure.

FIG. 2 shows a comparison result of the kit provided by the present disclosure and the foreign reagent.

### DETAILED DESCRIPTION OF THE EMBODIMENTS

The present disclosure provides a kit for detecting dust mite component-specific antibodies, including: a biotin-polystreptavidin-biotin-dust mite antigen-coated NC membrane, a washing solution, an ALP-labeled secondary antibody solution for dust mite component-specific antibodies, and a substrate solution,

where the biotin-polystreptavidin-biotin-dust mite antigen-coated NC membrane is formed by coating an NC membrane sequentially with biotin, polystreptavidin, and biotin-labeled dust mite antigens; the dust mite antigens include: dust mite protein Der f 1, dust mite protein Der f 2, dust mite protein Der p 1, dust mite protein Der p 2, dust mite protein Der p 5, dust mite protein Der p 7, dust mite protein Der p 10, dust mite protein Der p 11, dust mite protein Der p 20, dust mite protein Der p 21, dust mite protein Der p 23, dust mite protein Blo t 5, dust mite protein Blo t 10, dust mite protein Blo t 21, Dermatophagoides pteronyssinus, Dermatophagoides farinae, and Blomia tropicalis; and the dust mite antigens are coated on different positions on the NC membrane;

The dust mite component-specific antibodies include sIgE and sIgG, and the sIgG includes sIgG4.

In the present disclosure, there are no special limitations on the source of the dust mite antigens, and dust mite antigens conventionally synthesized with reference to conventional technical means may be used, such as dust mite antigens obtained by genetic engineering expression and purification or natural purification. In the present disclosure, with Der p 23 as an example, total RNA of Dermatophagoides pteronyssinus is used as a template and primers are designed and synthesized according to the sequence published by GenBank to amplify a Der p 23 coding gene by RT-PCR; a prokaryotic expression plasmid is constructed and transformed into Escherichia coli (E. coli), and IPTG is used to induce expression; and a target protein is collected and purified by column chromatography and then identified by SDS-PAGE and Western blot [Zhou Ying, Wu Meili, Zhu Hanting, Cui Yubao. Preparation of allergen Der p 23 recombinant protein and establishment of Der p 23-specific IgE chemiluminescence detection method [J]. Chinese Journal of Immunology, 2020, v.36 (20): 65-68.].

In the present disclosure, there are no special limitations on the method of biotin labeling, and the dust mite antigens can be labeled with biotin according to the biotin product specification. The biotin of the present disclosure may preferably be purchased from Sigma-Aldrich, with Item No.: B4501. In the present disclosure, the polystreptavidin may preferably be purchased from Yeasen BioTechnologies co., Ltd. (Shanghai), with Item No.: 35101ES03.

The biotin-polystreptavidin-biotin-dust mite antigen-coated NC membrane of the present disclosure may preferably be a biotin-polystreptavidin-biotin-dust mite antigen-coated NC membrane strip, hereinafter referred to as detection plate. In the present disclosure, a preparation method of the biotin-polystreptavidin-biotin-dust mite antigen-coated NC membrane may preferably include the following steps: coating biotin on an NC membrane, and drying; coupling polystreptavidin to the biotin, and drying; and coupling biotin-labeled dust mite antigens to the polystreptavidin. In the present disclosure, a high-accuracy automatic spotting instrument may preferably be used to coat biotin on the NC membrane. In the present disclosure, the drying may preferably be conducted at 37° C. In the present disclosure, after the biotin-labeled dust mite antigens are coated, an amplification structure of biotin-polystreptavidin-biotin-antigen is obtained. In the present disclosure, each dust mite antigen may be coated at a concentration preferably of 1 μg/mL to 1,000 μg/mL and more preferably of 20 μg/mL to 400 μg/mL, which is the optimal concentration obtained through experiments to ensure the accuracy of results.

In the present disclosure, the washing solution may preferably be a Tris-HCl buffer containing Tween-20, which can reduce non-specific adsorption. In the present disclosure, the Tween-20 may have a mass percentage preferably of 0.0004% to 0.02% and more preferably of 0.002% to 0.01% in the washing solution (referring to concentrated washing solution), thus ensuring the accuracy of results; and the washing solution may have a pH preferably of 7.2 to 7.6 and more preferably of 7.4. The washing solution of the present disclosure may be a concentrated solution, with a concentration factor of 25. In the present disclosure, a Tris-HCl buffer with a pH of 7.4 may preferably be used, which is added with 0.0004% to 0.02% of Tween-20 to obtain a washing solution, namely, a concentrated washing solution. The concentrated washing solution of the present disclosure may preferably be diluted with distilled water or deionized water in a volume ratio of 1:25 before use.

In the present disclosure, a preparation method of the ALP-labeled secondary antibody solution for dust mite component-specific antibodies may preferably include:

mixing ALP with a secondary antibody for dust mite component-specific antibodies to obtain the ALP-labeled secondary antibody solution for dust mite component-specific antibodies. In the present disclosure, the secondary antibody for dust mite component-specific antibodies may include an anti-human IgE antibody and an anti-human IgG antibody (including IgG4 antibody). In the present disclosure, the anti-human IgE antibody may preferably be purchased from American Abcam, with Item No.: ab195580. In the present disclosure, the anti-human IgG antibody may preferably be purchased from American Abcam, with Item No.: ab109489. In the present disclosure, the anti-human IgG4 antibody may preferably be purchased from American Abcam, with Item No.: ab238320. In the present disclosure, the secondary antibody solution for dust mite component-specific antibodies may have a mass concentration preferably of 0.1 μg/mL to 10 μg/mL and more preferably of 0.1 μg/mL to 5 μg/mL, thus ensuring the accuracy of results. In the present disclosure, the anti-human IgE antibody may preferably be a rabbit anti-human IgE antibody, namely, an antibody produced in a host rabbit that can specifically bind to human IgE. In the present disclosure, the anti-human IgG antibody may preferably be a rabbit anti-human IgG antibody, namely, an antibody produced in a host rabbit that can specifically bind to human IgG. In the present disclosure, the anti-human IgG4 antibody may preferably be a rabbit anti-human IgG4 antibody, namely, an antibody produced in a host rabbit that can specifically bind to human IgG4.

In the present disclosure, the substrate solution may preferably be a BCIP/NBT substrate solution, which is a mixed solution of nitroblue tetrazolium and 5-bromo-4-chloro-3-indolyl phosphate (NBT/BCIP). In the present disclosure, there are no special limitations on the source of the substrate solution, and a conventional commercially-available product well known to those skilled in the art may be adopted.

The kit of the present disclosure may quantitatively detect not noly the levels of 14 dust mite component-specific IgE antibodies in human serum at one time, which is clinically used for in vitro auxiliary diagnosis of type I hypersensitivity diseases, but also the levels of 14 dust mite component-specific IgG (including IgG4) antibodies in human serum, playing a role in clinical indication of dust mite desensitization treatment; and the kit may also detect a specific allergic component for a patient, which provides a basis for accurate immunotherapy on the patient. The kit of the present disclosure has reliable performance, high sensitivity, and wide linear range, and may be used in combination with full-automatic instruments (Hangzhou Zheda Dixun Biological Gene Engineering Co., Ltd., full-automatic western blotting instrument, model: DX-Blot 45, DX-Blot 45 II, and DX-AutoBlot 50), which may improve detection sensitivity and reliability and reduce cost. Specifically, there is no reagent for detecting various dust mite components in China, and the detection of dust mite components can only rely on imported reagents that are expensive. Compared with imported reagents (such as products of Thermo Fisher), the kit of present disclosure has a much lower price and involves a different method. In the present disclosure, the amount of antigen coated on the NC membrane is far lower than that of Thermo Fisher's products (about 1/1,000). For the products of Thermo Fisher, the detection of one item requires one CAP and each CAP requires 40 μL of test serum, while the present disclosure only requires 300 μL of test serum to detect 17 items at a time, with 17.6 μL of serum for each item on average.

In the present disclosure, biotin, polystreptavidin, and biotin-coupled dust mite antigens are coated on an NC membrane. When serum with dust mite-specific antibodies sIgE or sIgG (including IgG4) is added to the detection plate, the antibodies sIgE or sIgG (including IgG4) bind to the antigens fixed on the membrane such that an immune complex of biotin-polystreptavidin-biotin-antigen-sIgE or sIgG (including IgG4) is formed and fixed on the membrane, and the remaining unbound substances are removed by washing; then an ALP-anti-human sIgE antibody complex is added to the detection plate to form an immune complex of biotin-polystreptavidin-biotin-antigen-sIgE-anti-human IgE antibody-ALP or an immune complex of biotin-polystreptavidin-biotin-antigen-sIgG-anti-human IgG antibody-ALP (including an immune complex of biotin-polystreptavidin-biotin-antigen-sIgG4-anti-human IgG4 antibody-ALP), and the remaining unbound substances are removed by washing; and then a substrate solution is added to conduct a chromogenic reaction with the enzyme, where a color intensity of a line/dot represents an sIgE or sIgG (including IgG4) concentration in the serum.

A method for high-throughput detection of dust mite antigens and single-component sIgE or sIgG (including IgG4) using the kit of the present disclosure may preferably include the following steps:

wetting the detection plate with a working washing solution; adding test serum, and incubating; washing, adding the ALP-labeled secondary antibody solution for dust mite component-specific antibodies, and incubating; washing, adding the substrate solution, and incubating; and stopping the reaction, drying, and scanning with a scanner.

In the present disclosure, the detection plate is wet with a working washing solution. In the present disclosure, In some embodiments, the detection plate is taken out and added with 100 μL to 500 μL of a working washing solution, and then placed on a mixer for thorough wetting, and then the working washing solution is poured out. The working washing solution of the present disclosure may preferably be a working washing solution obtained by diluting the aforementioned concentrated washing solution.

In the present disclosure, after the detection plate is wet, test serum is added and incubated. In the present disclosure, 100 μL to 500 μL of serum may preferably be added to the detection plate and then incubated on a mixer at room temperature, and the incubation may be conducted preferably for 30 min to 60 min.

In the present disclosure, after the serum is added and incubated, the plate is washed, and then the ALP-labeled secondary antibody solution for dust mite component-specific antibodies is added and incubated. In the present disclosure, In some embodiments, the liquid in the detection plate may be poured out, and the detection plate may be rinsed with a working washing solution 3 to 5 times, with 10 s to 30 s for each time. In the present disclosure, In some embodiments, 100 μL to 500 μL of a secondary antibody working solution may preferably be added to the detection plate and then incubated on a mixer at room temperature for 30 min to 60 min.

In the present disclosure, after the binding of the secondary antibody, the detection plate is washed, and then the substrate solution is added and incubated; the reaction is stopped, and the plate is dried; and the plate is scanned with a scanner. In the present disclosure, In some embodiments, the liquid in the detection plate may be poured out, and the detection plate may be rinsed with a working washing solution 3 to 5 times, with 10 s to 30 s for each time. In the present disclosure, In some embodiments, 100 μL to 500 μL of a substrate solution may preferably be added to the detection plate and then incubated on a mixer at room temperature for 10 min to 30 min. The detection plate is rinsed with running water to stop the enzyme reaction. After the detection plate is dried, the membrane strip is scanned with a scanner, and results are read with analysis software. The scanning analysis software of the present disclosure may preferably be application software that can analyze and calculate a corresponding sIgE or sIgG (including IgG4) concentration according to a color intensity at a protein site (Hangzhou Zheda Dixun Biological Gene Engineering Co., Ltd., allergen semi-quantitative analysis software). The protein site refers to a site coated with a dust mite antigen on the membrane strip. The concentration refers to a content level of sIgE or sIgG (including IgG4) in serum, with an international unit of IU/mL or U/mL.

The kit for detecting dust mite component-specific antibodies sIgE according to the present disclosure will be further described in detail below with reference to specific examples. The technical solutions of the present disclosure include, but are not limited to, the following examples.

### Example 1

A kit for detecting dust mite components based on an anti-human IgE antibody included the following components:

detection plate: a biotin-polystreptavidin-biotin-dust mite antigen-coated NC membrane strip;

concentrated washing solution (25×): concentrated Tris buffer;

IgE secondary antibody working solution: ALP-labeled anti-human IgE antibody solution; and

substrate solution: B CIP/NB T.

Preparation of the Detection Plate:

A high-accuracy automatic spotting instrument was first used to coat biotin (Sigma-Aldrich, Item No.: B4501) on an NC membrane strip, and then the strip was dried at 37° C.; then polystreptavidin (Yeasen BioTechnologies co., Ltd. (Shanghai), Item No.: 35101E503) was coated, and then the strip was dried at 37° C.; and then 14 biotin-labeled dust mite proteins (Der f 1, Der f 2, Der p 1, Der p 2, Der p 5, Der p 7, Der p 10, Der p 11, Der p 20, Der p 21, Der p 23, Blo t 5, Blo t 10, and Blo t 21) and 3 dust mite allergens (Dermatophagoides pteronyssinus, Dermatophagoides farinae, and Blomia tropicalis) were coated to form an amplification structure of biotin-polystreptavidin-biotin-antigen. The dust mite antigens were coated at a concentration of 1 μg/mL to 1,000 μg/mL.

Preparation of the Washing Solution:

A Tris-HCl buffer with a pH of 7.4 was added with 0.008% of a surfactant (Tween-20) to obtain a concentrated washing solution. The concentrated washing solution was diluted with distilled water or deionized water in 1:25 to obtain a working washing solution.

Preparation of the IgE secondary antibody working solution:

ALP was mixed with an anti-human IgE antibody (American Abcam, Item No.: RM122) to obtain an ALP-labeled anti-human IgE antibody solution with a concentration of 4 μg/mL. The anti-human IgE antibody was a rabbit anti-human IgE antibody.

Preparation of the Substrate Solution:

Nitroblue tetrazolium and 5-bromo-4-chloro-3-indolyl phosphate were mixed to obtain the substrate solution (NBT/BCIP).

### Example 2

A dust mite component-specific IgE detection kit (protein microarray method) included the following components:

(1) detection plate: a biotin-polystreptavidin-biotin-dust mite antigen-coated NC membrane strip, for 20 persons;

(2) IgE secondary antibody working solution: ALP-labeled anti-human IgE antibody solution (ALP labeling was conducted by a conventional labeling method), specification: 8 mL;

(3) substrate solution: BCIP/NBT, specification: 8 mL;

(4) concentrated washing solution (25×): concentrated Tris buffer, specification: 20 mL;

(5) instructions: the basic information, detection principle, and operation process of the kit; and

(6) colourimetric card: which is used to compare with protein sites.

The colourimetric card was isometrically made according to the spotting positions, which can be used for preliminary position and color intensity comparison.

### Example 3

Serum was collected from dust mite allergic patients, and then detected by the kit of the present disclosure according to the following detection steps:

1. The detection plate was taken out and added with 300 μL of the working washing solution, and then placed on a mixer for thorough wetting, and then the working washing solution was poured out.

2. 300 μL of serum was added to the detection plate and then incubated for 30 min on a mixer at room temperature.

3. The solution in the detection plate was poured out, and the detection plate was rinsed with a working washing solution 5 times, with 15 s for each time.

4. 300 μL of the IgE secondary antibody working solution was added to the detection plate and then incubated for 30 min on a mixer at room temperature.

5. The liquid in the detection plate was poured out, and the detection plate was rinsed with a working washing solution 5 times, with 15 s for each time.

6. 300 μL of the substrate solution was added to the detection plate and then incubated for 15 min on a mixer at room temperature.

7. The detection plate was rinsed with running water to stop the enzyme reaction.

8. The detection plate was dried, and results were read with scanning analysis software.

Results were shown in Table 1. It could be seen that the dust mite allergic patient 1 was positive for Dermatophagoides pteronyssinus, Dermatophagoides farinae, and Blomia tropicalis sIgE; and the dust mite allergic patient 1 was positive for dust mite proteins Der 1, Der f 2, Der p 1, Der p 2, Der p 10, Der p 21, and Blo t 5, but negative for the remaining dust mite proteins. Therefore, the specific allergic components can be detected for a patient, providing basis for the administration of precise immunotherapy to patients. The dust mite allergic patient 2 was positive for Dermatophagoides pteronyssinus, negative for Dermatophagoides farinae, and Blomia tropicalis, and positive only for dust mite proteins Der p 1, Der p 2. Therefore, Therefore, the desensitization treatment can be targeted with desensitization preparations containing only Der p 1 and Der p 2. The dust mite allergic patient 3 was positive for Dermatophagoides pteronyssinus and Dermatophagoides farinae-specific sIgE, negative for Blomia tropicalis, weak-positive for the dust mite protein Der p 2, and strong-positive for the dust mite protein Der p 10. Because Der p 10 was a purified single-component protein, its positive grade was higher than that of the crude extracts of Dermatophagoides pteronyssinus and Dermatophagoides farinae. It also showed that the dust mite allergy of the patient was mainly caused by the dust mite protein Der p 10.

TABLE 1
Serum sIgE detection results of dust mite allergic patients

| | Patient No. | | |
|---|---|---|---|
| | Dust mite | Dust mite | Dust mite |
| Item name (unit: | allergic | allergic | allergic |
| IU/mL) | patient 1 | patient 2 | patient 3 |
| Dermatophagoides | 39.28 | 3.49 | 14.55 |
| pteronyssinus | | | |
| Dermatophagoides | 56.12 | 0.14 | 16.88 |
| farinae | | | |
| Blomia tropicalis | 0.74 | 0.27 | 0.17 |
| Der p 1 | 4.21 | 1.53 | 0.15 |
| Der f 1 | 2.33 | 0.02 | 0.00 |
| Der p 2 | 1.35 | 0.49 | 0.41 |
| Der f 2 | 0.98 | 0.33 | 0.17 |
| Der p 5 | 0.33 | 0.22 | 0.00 |
| Der p 7 | 0.15 | 0.19 | 0.10 |
| Der p 10 | 0.60 | 0.25 | 29.90 |
| Der p 11 | 0.31 | 0.05 | 0.24 |
| Der p 20 | 0.21 | 0.08 | 0.28 |
| Der p 21 | 0.41 | 0.16 | 0.09 |
| Der p 23 | 0.09 | 0.27 | 0.00 |
| Blo t 5 | 0.39 | 0.19 | 0.08 |
| Blo t 10 | 0.17 | 0.19 | 0.11 |
| Blo t 21 | 0.21 | 0.17 | 0.34 |

The above detection results were graded according to the relationship between specific IgE concentrations and grading standards internationally. A detection result ≥0.35 IU/mL indicated that an allergen-specific antibody sIgE bound to an allergen, and a detection result <0.35 IU/mL indicated that an allergen-specific antibody sIgE concentration was not detectable or very low.

The relationship between specific IgE concentrations and grading standards internationally was shown in Table 2:

TABLE 2
Relationship between specific IgE concentrations
and grading standards

| International specific IgE | |
|---|---|
| concentration (IU/mL) | International grading standard |
| <0.35 | 0 (negative) |
| 0.35 to 0.70 | 1 (weak positive) |
| 0.71 to 3.50 | 2 (positive) |
| 3.51 to 17.50 | 3 (relatively-strong positive) |
| 17.51 to 50.0 | 4 (strong positive) |
| 50.01 to 100.0 | 5 (extra-strong positive) |
| >100.0 | 6 (extremely-strong positive) |

### Example 4

Serum was collected from patients with allergic asthma and rhinitis that underwent dust mite SIT, and then detected by the kit of the present disclosure according to the following detection steps:

1. The detection plate was taken out and added with 300 μL of the working washing solution, and then placed on a mixer for thorough wetting, and then the working washing solution was poured out.

2. 300 μL of serum was added to the detection plate and then incubated for 30 min on a mixer at room temperature.

3. The liquid in the detection plate was poured out, and the detection plate was rinsed with a working washing solution 5 times, with 15 s for each time.

4. 300 μL of the IgG4 secondary antibody working solution was added to the detection plate and then incubated for 30 min on a mixer at room temperature.

5. The liquid in the detection plate was poured out, and the detection plate was rinsed with a working washing solution 5 times, with 15 s for each time.

6. 300 μL of the substrate solution was added to the detection plate and then incubated for 15 min on a mixer at room temperature.

7. The detection plate was rinsed with running water to stop the enzyme reaction.

8. The detection plate was dried, the membrane strip was scanned with a scanner, and results were read with analysis software.

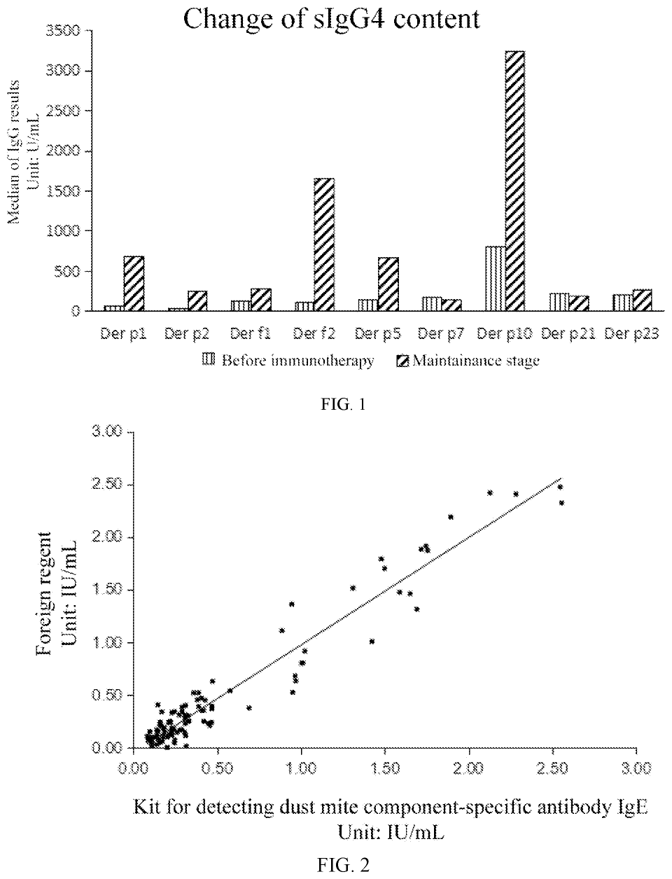

The results were shown in FIG. 1 (FIG. 1 shows sIgG4 content detection results), and it could be seen that, at the maintenance phase, the levels of Der p 1, Der p 2, Der f 1, Der f 2, Der p 5, Der p 10, and Der p 23-specific sIgG4 antibodies were higher than that before the immunotherapy. It showed that the level of dust mite sIgG4 continued to rise during the immunotherapy. Therefore, IgG4 is a useful index for evaluating the clinical responses of immunotherapy.

### Comparative Example 1

The dust mite component-specific IgE detection kit (protein microarray) of the present disclosure and the ImmunoCAP from Thermo Fisher were both used to detect 100 cases of sera, and the following results were obtained (Table 3, and FIG. 2) (FIG. 2 shows a comparison result of the present disclosure and the foreign agent):

TABLE 3
Comparison of detection results

| | Immuno CAP | | | |
|---|---|---|---|---|
| | | Negative | Positive | Total |
| Product of the present disclosure | Negative | 57 | 3 | 60 |
| | Positive | 3 | 40 | 43 |
| | Total | 60 | 43 | 103 |

Coincidence rate for positive results=40/43=93.02% (95% CI 79.88% to 98.18%)

Coincidence rate for negative results=57/60=95.00 (95% CI 85.18% to 98.70%)

Total coincidence rate=(57+40)/103=94.17% (95% CI 87.62% to 97.55%)

Correlation coefficient r=0.9665

It could be seen from the results that, compared with the existing dust mite component detection products, the present disclosure had a higher coincidence rate; the detection results of the foreign reagent for the 100 serum samples showed a very high correlation with that of the present disclosure; and with the existing products, one CAP may only detect one antigen, while the present disclosure may detect 14 dust mite proteins and 3 dust mite allergens at one time.

### Comparative Example 2

The plate was prepared by a method without using the “biotin-poly polystreptavidin-biotin-antigen” amplification structure: 14 dust mite protein fractions and 3 dust mite antigens were directly coated on nitrocellulose membrane strips using an automatic spotting instrument, while the other working solutions were prepared in the same way. The detection results by the detection plate prepared by this method and that of the present disclosure are shown in Table 4.

TABLE 4
Detection results of serum sIgG4 in dust mite allergic patients

| | Plate without the | | Plate of the | |
|---|---|---|---|---|
| | amplification structure | | present disclosure | |
| | Dust mite | Dust mite | Dust mite | Dust mite |
| Item name (unit: | allergic | allergic | allergic | allergic |
| IU/mL) | patient 1 | patient 2 | patient 1 | patient 2 |
| Dermatophagoides | 653.2 | 237.2 | 763.5 | 366.1 |
| pteronyssinus | | | | |
| Dermatophagoides | 152 | 284.8 | 168.1 | 405.4 |
| farinae | | | | |
| Blomia tropicalis | 13.5 | 36.4 | 15.4 | 39.1 |
| Der p 1 | 84.6 | 233.7 | 85.9 | 378.9 |
| Der f 1 | 10.3 | 203.9 | 14.3 | 224.3 |
| Der p 2 | 0.01 | 226.4 | 1.23 | 365.6 |
| Der f 2 | 13.3 | 382.6 | 21.3 | 501.3 |
| Der p 5 | 4.61 | 146.4 | 8.6 | 149.8 |
| Der p 7 | 226.1 | 80.9 | 321.1 | 90.2 |
| Der p 10 | 11 | 87.8 | 15.6 | 89.1 |
| Der p 11 | 32.1 | 21.5 | 43.6 | 26.4 |
| Der p 20 | 16.4 | 0.13 | 21.7 | 1.03 |
| Der p 21 | 398.4 | 39.9 | 509.7 | 56.7 |
| Der p 23 | 22 | 265.5 | 35.1 | 377.8 |
| Blo t 5 | 0.13 | 15.6 | 0.24 | 16.7 |
| Blo t 10 | 1.56 | 2.33 | 2.01 | 2.36 |
| Blo t 21 | 14.6 | 2.11 | 15.5 | 2.99 |

In the present disclosure, based on the threshold value determined by the ROC curve of the clinical trial, the reference value for IgG4 negativity and positivity was determined to be 250 U/mL, with results of ≥250 U/mL considered positive and results <250 U/mL considered negative. As can be seen from Table 4, the detection values of the present disclosure with the amplified structure were all improved, while the detection results of the dust mite protein Der p 7 in dust mite allergic patient 1 were false negative on the membrane strip without the amplification structure; and the detection results of the Dermatophagoides pteronyssinus, Der p 1, and Der p 2 in dust mite allergic patient 2 were false negative on the membrane strip without the amplification structure. Therefore, as can be seen, the amplification structure allows the antigen to react fully with the antibody, which may improve the sensitivity of the determination and reduce the false negative results.

The above descriptions are merely preferred implementations of the present disclosure. It should be noted that a person of ordinary skill in the art may further make several improvements and modifications without departing from the principle of the present disclosure, but such improvements and modifications should be deemed as falling within the protection scope of the present disclosure.

1-8. (canceled)

9. A kit for detecting dust mite component-specific antibodies, comprising: a biotin-polystreptavidin-biotin-dust mite antigen-coated nitrocellulose (NC) membrane, a washing solution, an alkaline phosphatase (ALP)-labeled secondary antibody solution for dust mite component-specific antibodies, and a substrate solution;
wherein the biotin-polystreptavidin-biotin-dust mite antigen-coated NC membrane is formed by coating an NC membrane sequentially with solidified biotin, polystreptavidin, and biotin-labeled dust mite antigens; the dust mite antigens comprise: dust mite protein Der f 1, dust mite protein Der f 2, dust mite protein Der p 1, dust mite protein Der p 2, dust mite protein Der p 5, dust mite protein Der p 7, dust mite protein Der p 10, dust mite protein Der p 11, dust mite protein Der p 20, dust mite protein Der p 21, dust mite protein Der p 23, dust mite protein Blo t 5, dust mite protein Blo t 10, dust mite protein Blo t 21, Dermatophagoides pteronyssinus, Dermatophagoides farinae, and Blomia tropicalis; and the dust mite antigens are coated at different positions on the NC membrane;
the dust mite component-specific antibodies comprise sIgE and sIgG, and sIgG comprises sIgG4;
a preparation method of the biotin-polystreptavidin-biotin-dust mite antigen-coated NC membrane comprises the following steps: coating biotin on an NC membrane, and drying; coupling polystreptavidin to biotin, and drying; and coupling biotin-labeled dust mite antigens to polystreptavidin; and
a preparation method of the ALP-labeled secondary antibody solution for dust mite component-specific antibodies comprises:
mixing ALP with a secondary antibody for dust mite component-specific antibodies to obtain the ALP-labeled secondary antibody solution for dust mite component-specific antibodies.

10. The kit according to claim 9, wherein the washing solution is a Tris-HCl buffer containing Tween-20.

11. The kit according to claim 10, wherein Tween-20 has a mass percentage of 0.0004% to 0.02% in the washing solution, and the washing solution has a pH of 7.2 to 7.6.

12. The kit according to claim 9, wherein the secondary antibody solution for dust mite component-specific antibodies has a mass concentration of 0.1 μg/mL to 10 μg/mL.

13. The kit according to claim 9, wherein the substrate solution is a BCIP/NBT substrate solution.
